(19) United States
(12) Patent Application Publication
(10) Pub. No.: US 2013/0240638 A1
(43) Pub. Date: Sep. 19, 2013

(54) ENHANCED MICRO PARTICLES DRUG AND ODOR DIFFUSION APPARATUS

(71) Applicants: Cem Kaplan, Istanbul (TR); Ertan Korkmaz, Istanbul (TR)

(72) Inventors: Cem Kaplan, Istanbul (TR); Ertan Korkmaz, Istanbul (TR)

(73) Assignee: CEKK KIMY A VE KOZMETIK URUNLERI SAN VE DIS TIC LTD. STI, Istanbul (TR)

(21) Appl. No.: 13/814,099

(22) PCT Filed: Jan. 10, 2011
(86) PCT No.: PCT/TR2011/000005

§ 371 Date: May 21, 2013

(30) Foreign Application Priority Data
Aug. 9, 2010 (TR) 2010/06515

(51) Int. Cl. A24F 25/00; A61L 9/14

(52) U.S. Cl. CPC A61L 9/14
USPC 239/34

(57) ABSTRACT

A diffusion apparatus for micro particles, drugs and odor is provided. The upper and middle diffusion parts have been combined and the silencer and the air inlet and the upper diffusion chamber that are required for the diffusion model have been put together in one piece.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

REFERENCE TO AN APPENDIX SUBMITTED ON COMPACT DISC

Not applicable.

### BACKGROUND OF THE INVENTION

1. Field of the Invention

The invention is about a diffusion apparatus for micro particles drugs and odor.

2. Description of Related Art Including Information Disclosed Under 37 CFR 1.97 and 37 CFR 1.98

The models that have been used for odors for the present operate on aeoral spraying systems. With the enhanced and used model, the odors are divided into micro particles and thereby they are provided the advantage of being suspended in the air and travel for a longer period of time. With this model that have been enhanced the last, the parts of the diffusion apparatus have been divided into two parts on the bottle and the model and the design is undertaken such that under conditions of every connection, a perfect combination is provided and after the liquid that has been used with the special cap and bottle is finished, the same is disposed of and a new bottle could be mounted. Hence the whole model is combined in one part.

### BRIEF SUMMARY OF THE INVENTION

The new model had been designed by developing the product that had been registered with the number 2009/03805 in the name of our firm.

The micro particle diffusion apparatus that has been mentioned in our utility model with the number 2009/03805; consists of a micro particle apparatus, a sleeve, a crank, a plastic pipe and a colliding plate. At the same time, there is a micro particle diffusion protector that protects the micro particle diffusion apparatus and that has a particle outlet.

To diffuse liquids such as drugs, essences, natural oil, insecticides in terms of their essences and chemistry, without using the spraying and heating, to diffuse the said liquids particles that are contained in a bottle by using air at a certain pressure, by engaging in the vacuum system, by giving air at a certain pressure, by vacuuming the liquid inside the bottle into the apparatus, and by colliding the same on to the colliding plate, without damaging the essence of the liquid, by following the path of the plastic pipe, the micro particle apparatus, the crank and the sleeve; from the particle outlet that has been located on the micro particle diffusion apparatus protector; to let out the micro particles.

### BRIEF DESCRIPTION OF THE SEVERAL VIEWS OF THE DRAWINGS

The parts that have been used in this model have been divided into three parts in the newly enhanced model; and all of the parts have been combined into establishing a single model; as

a. upper diffusion

b. medium diffusion and

c. the lower diffusion.

The figures that are going to help with the explanation of the invention;

FIG. 1: Enhanced micro particle diffusion bottle and apparatus—the whole

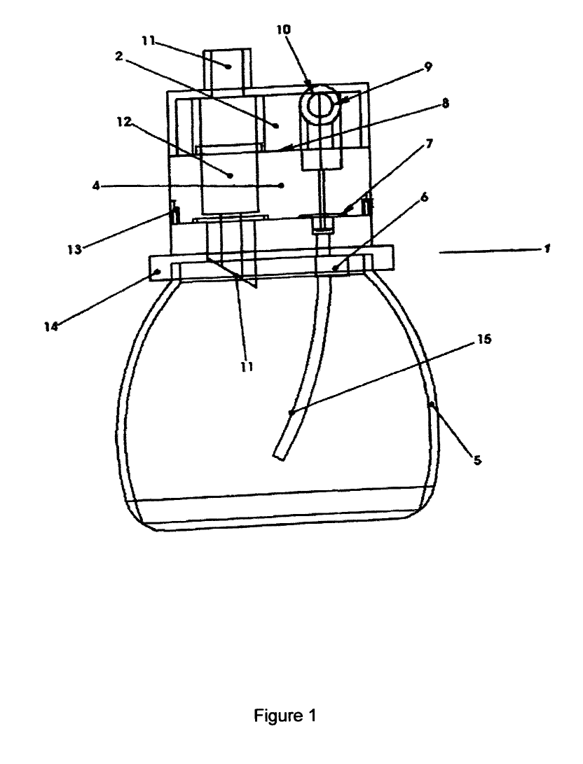

FIG. 2: Enhanced micro particle diffusion apparatus—the upper part

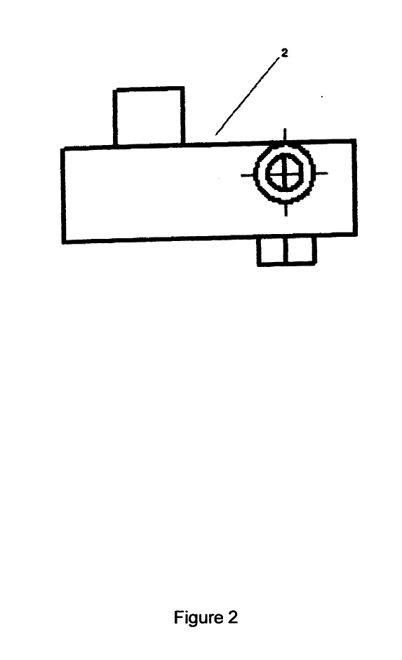

FIG. 3: Enhanced micro particle diffusion apparatus—the medium part

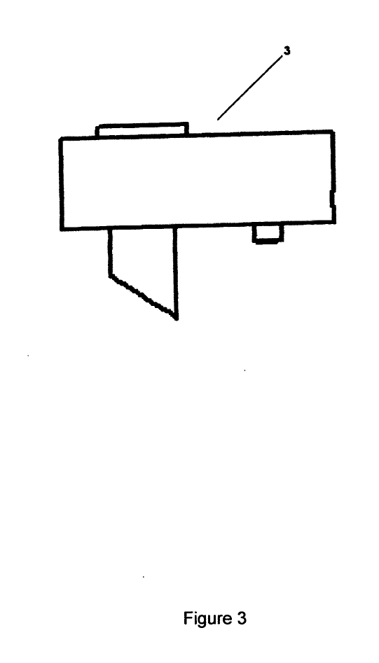

FIG. 4: Enhanced micro particle diffusion apparatus—the lower part

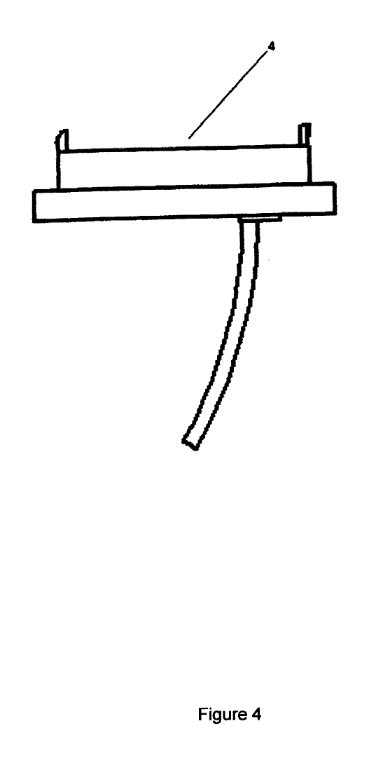

FIG. 5: Enhanced micro particle diffusion bottle and apparatus—the side view

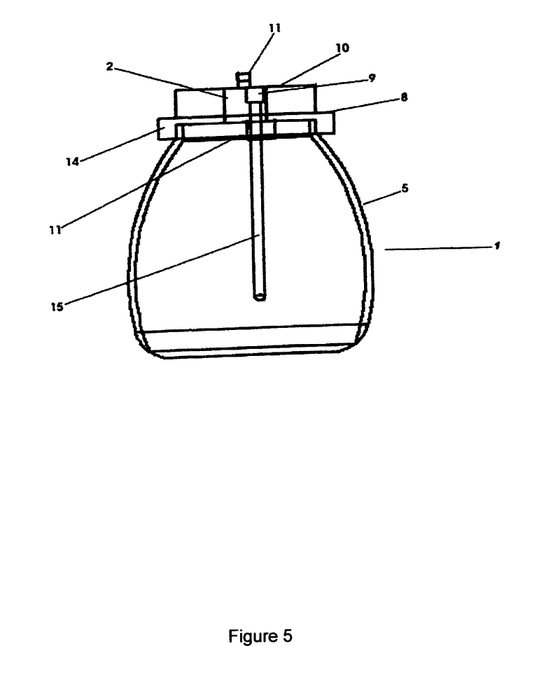

FIG. 6: Enhanced micro particle diffusion apparatus—the top view

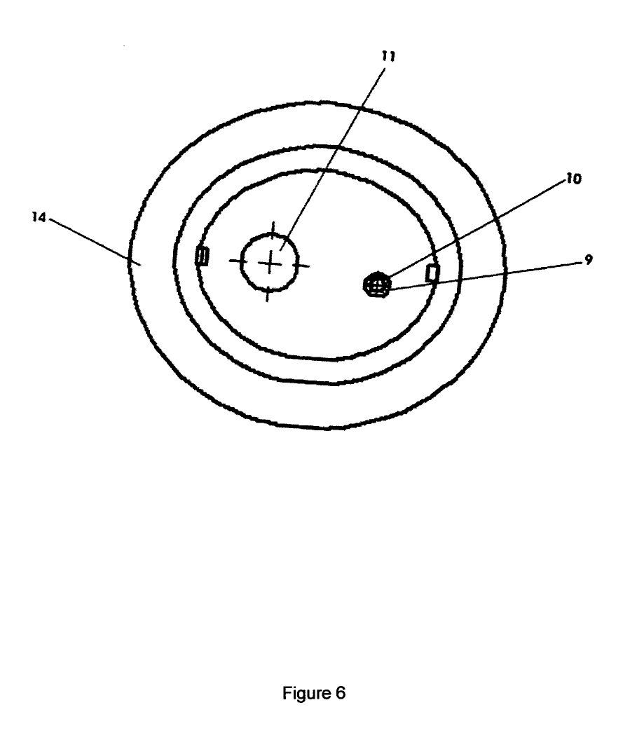

FIG. 7: Enhanced micro particle diffusion bottle and apparatus—the cross section of the side view

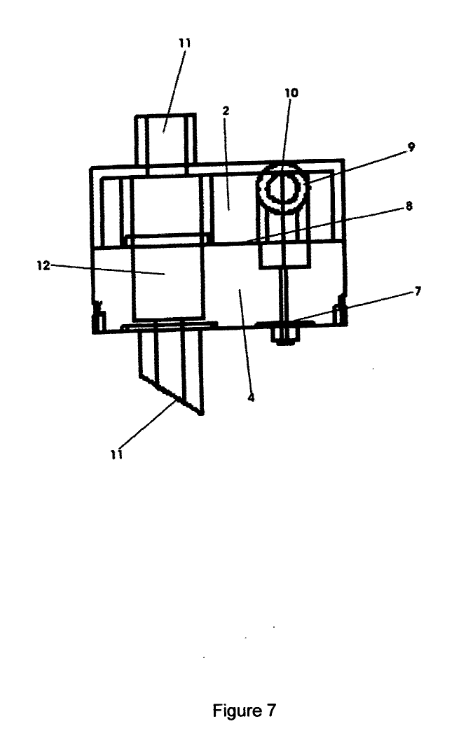

FIG. 8: Enhanced micro particle diffusion bottle and apparatus—the cross section of the top view

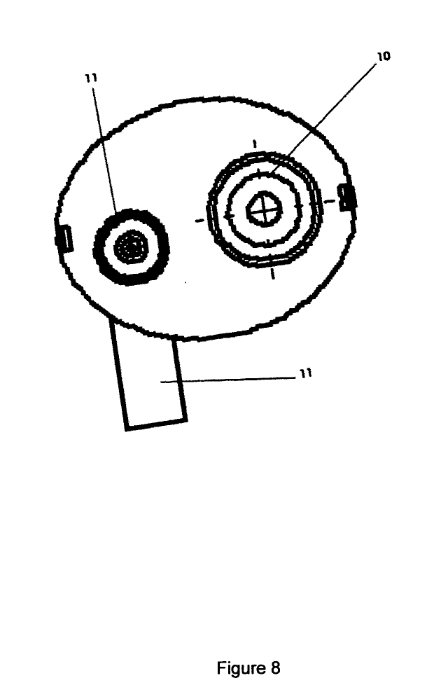

FIG. 9: Enhanced micro particle diffusion bottle and apparatus—the whole view from perspective.

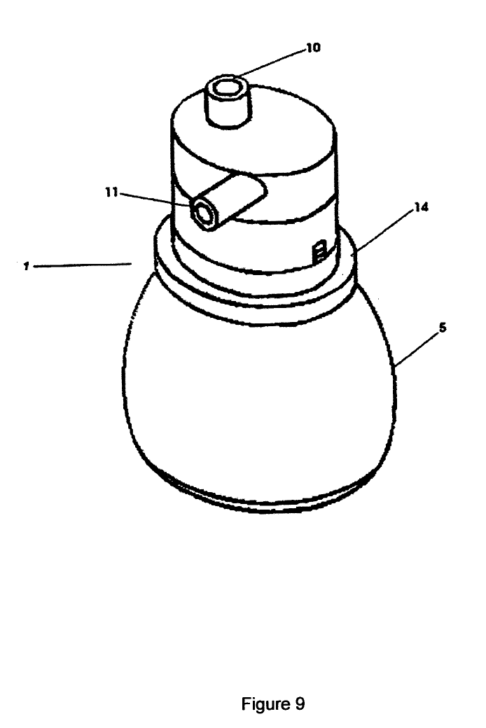

The names of the parts that are referred to within the figures;

1—The whole of the micro particle diffusion apparatus

2—The upper part of the micro particle diffusion apparatus

3—The medium part of the micro particle diffusion apparatus

4—The lower part of the micro particle diffusion apparatus

5—The lower part of and the bottle of the micro particle diffusion apparatus

6—The lower diffusor diffusion lower chamber of the micro particle diffusion apparatus

7—The upper diffusor diffusion upper chamber of the micro particle diffusion apparatus

8—The connection point of the apparatus

9—The crank

10—The air inlet

11—The air outlet

12—The silencer

13—The holding claws

14—The cap of the bottle

15—The suction pipe

### DETAILED DESCRIPTION OF THE INVENTION

The invention is about a diffusion apparatus for micro particles drugs and odour. The model consists of a whole of the micro particle diffusion apparatus (1), the upper part of the micro particles diffusion apparatus (2), the middle part of the micro particles diffusion apparatus (3), the lower part of the micro particles diffusion apparatus (4), the lower part of and the bottle of the micro particle diffusion apparatus (5), the lower diffusor diffusion lower chamber of the micro particle diffusion apparatus (6), the upper diffusor diffusion upper chamber of the micro particle diffusion apparatus (7), the connection point of the apparatus (8), the crank (9), the an inlet (1), the air outlet (11), the silencer (12), the bottling, claws (13), the cap of the bottle (14), and a suction pipe (15).

In our invention, the upper and middle diffusion parts have been combined and the silencer (12) and the air inlet (10) and the upper diffusion chamber (7) that are required for the diffusion model have been put together in one piece.

Besides this because of the bottle top tight bottle cap (14) that has been newly developed, the lower diffusion apparatus (4) had been provided on the bottle (5) and the lower diffusion apparatus (3) along with the middle diffusion apparatus (3) had been joined at every use of the bottle (5); and a new diffusion had been provided for each time of the use. Accordingly, the user's bodies are kept away from the splashes of the liquid for every use, and after the odour or the drug that has been used is finished, this is going to be disposed off and a new bottle is going to be placed and the said diffusion model is provided for every time of the use.

Because of the whole of the apparatus (1) that we had developed, the lower diffusion apparatus (4) that is with the bottle (5) for it to be fixed tightly in every use, both in the lower diffusion apparatus (4) and in the middle diffusion apparatus (3) there are bolding claws (13) and the same is safely and tightly holded with the said material. When the user wants to disconnect the bottle (5), it is going to be enough to push on to the holding claws (13) that are located on the side of the whole of the diffusion apparatus (1).

With the diffusion model that has been developed by us, the air that has been given inside through the top sleeve part reaches the chamber (7) of the upper diffusion apparatus that has been located in the middle portion and from there joins with the lower division chamber (6) that is located in the lower diffusion apparatus (4) diffused the liquid that has been used within the bottle (5) and thus diffuses the same into the medium through the hole of the silencer (12).

The micro diffusion apparatus of this model could be alternatively connected from different parts;

1—The micro diffusion model could be used as a whole. The product that has been sold in its joined condition, could be supported with kind of air inlet and could be used on its own. In this manner a disposable function could be provided.

2—The micro diffusion model and all of the other parts that have been shown in the figures could be fitted inside the cap of the bottle. It is possible for the same to be provided inside the bottle. In this manner also, a disposable function could be provided.

3—With the fixing of the micro diffusion model as a whole inside the bottle completely the same could be fixed, and the silencer and the air outlet could be considered as a different model.

4—The insertion points of the bottles could be supported with a screw system, tight fixing and special locking systems, instead, of the claws.

5—It is possible to let out the micro particles by way of air from any directions.

6—The form of the bottle could be provided in any shape or design that has been desired.

7—The whole condition of the micro diffusion apparatus could be manufactured in the forms of a circle, a square, and a rectangle instead of the elliptical shape. It is of a characteristic that could be manufactured out of all kinds of different raw materials and materials.

1. A micro particle drug and odor diffusion apparatus comprises: the whole of a micro particle diffusion apparatus, an upper part of the micro particles diffusion apparatus, a middle part of the micro particles diffusion apparatus, a lower part of the micro particles diffusion apparatus, a lower part of and the bottle of the micro particle diffusion apparatus, a lower diffusor diffusion lower chamber of the micro particle diffusion apparatus, an upper diffusor diffusion upper chamber of the micro particle diffusion apparatus, a connection point of the apparatus, a crank, an air inlet, an air outlet, a silencer, holding claws, a cap of the bottle, and a suction pipe.

2. The diffusion apparatus of claim 1, wherein the upper diffusion the middle diffusion and the lower diffusion parts that make up a single part by joining to each other; it has been provided to be in connection with and joining in a perfect manner and the dispose off after the liquid that has been used is finished, and in a shape that a new bottle could be mounted that divides into two pieces in the said manner, the whole of the diffusion apparatus, the special bottle and the bottle cap.

3. The diffusion apparatus of claim 2, wherein the silencer and the air inlet and the upper diffusion part chamber are required for the diffusion model that becomes a single part by joining the upper and middle diffusion parts.

4. The diffusion apparatus of claim 1, wherein the lower diffusion apparatus is provided within the bottle and the lower diffusion apparatus along with the middle diffusion apparatus are joined at every use of the bottle and a new diffusion is provided for each use and it comprises of the bottle top tight bottle cap to provide the said function.

5. The diffusion apparatus of claim 1, wherein because of the whole of the apparatus, the lower diffusion apparatus that is along with the bottle for it to fit at every use tightly, there are holding claws both on the middle diffusion apparatus and on the lower diffusion apparatus so that a safe and tight hold is provided.

6. The diffusion apparatus of claim 1, wherein the top sleeve portion from where air is given with the developed diffusion model, the chamber of the upper diffusion apparatus that is located in the middle section where the air reaches, the lower part chamber that has been located on the lower diffusion apparatus and the hole of the silencer that distributes the used liquid into the medium by way of diffusion inside the bottle.
